(19) United States
(12) Patent Application Publication
(10) Pub. No.: US 2005/0186647 A1
(43) Pub. Date: Aug. 25, 2005

(54) Methods for identifying or monitoring a patient with increased risk of cardiovascular calcification

(76) Inventor: Ping Gao, San Diego, CA (US)

Correspondence Address: DAVID R PRESTON & ASSOCIATES APC, 12625 HIGH BLUFF DRIVE, SUITE 205, SAN DIEGO, CA, 92130, US

(21) Appl. No.: 10/987,513

(22) Filed: Nov. 12, 2004

Related U.S. Application Data
(60) Provisional application No. 60/519,450, filed on Nov. 12, 2003.

(51) Int. Cl. G01N 33/53; G01N 33/537; G01N 33/543

(52) U.S. Cl. 435/7.93

(57) ABSTRACT

The present invention includes novel methods of using a calcification risk index (CRI) for identifying or monitoring a patient with increased risk of cardiovascular calcification and/or mortality, or monitoring an effect of therapeutic treatment for a patient having cardiovascular calcification or with increased risk of cardiovascular calcification. The methods may include determining an alpha-2 HS glycoprotein level or circulating calcification inhibitory capacity in a biological sample, and comparing the level to at least one of the parameters selected from a calcium level, a phosphate level and a calcium×phosphate product level, or the combination of these values. The present invention may allow one able to identify or monitor a patient having increased risk of cardiovascular calcification and/or predicting mortality, or to monitor an effect of treatment for a patient having an increased risk of cardiovascular calcification or a patient who already has cardiovascular calcification.

### CROSS REFERENCE TO RELATED APPLICATIONS

The present application claims benefit of priority to U.S. Provisional Patent Application Ser. No. 60/519,450 filed on Nov. 12, 2003, entitled, “Methods for Identifying and Monitoring Patients with Increased Risk of Cardiovascular Calcification,” naming Ping Gao as inventor, and is herein incorporated by reference in its entirety.

### TECHNICAL FIELD

The present invention includes novel methods for identifying or monitoring a patient with increased risk of cardiovascular calcification, or for monitoring an effect of a therapeutic treatment for a patient with increased risk of cardiovascular calcification or a patient who already has cardiovascular calcification. More specifically, the present invention includes determining a alpha-2 HS glycoprotein (AHSG) level or circulating calcification inhibitory capacity in a biological sample, and comparing the level or capacity to at least one of the parameters such as a calcium level, a phosphate level and a calcium×phosphate product level, or a combination of one, or more of these values and determining the patient has or is at risk of developing cardiovascular calcification if the patient's comparison value is different from a control value or a normal population.

### BACKGROUND

The clinical need for identifying or monitoring a patient having or at risk of developing cardiovascular calcification or other soft tissue calcification is well documented. It is also extremely important clinically that one can monitor the effects of a therapeutic treatment for a patient who either already has a cardiovascular calcification or has an increased risk of cardiovascular calcification, whether or not an alternative examination indicated the existence of such condition. By doing so, one may monitor, control or slow down the progression of calcification, which may predict or reduce cardiac morbidity or mortality.

There are several groups or populations that may have an increased risk of cardiovascular calcification such as but not limited to patients with diabetes, obesity, rheumatoid arthritis, chronic liver diseases such as but not limited to hepatitis, liver cirrhoses, etc., as well as an aged population. It is also known that cardiovascular calcification and cardiovascular mortality is greatly increased in patients with chronic renal failure, especially for those end-stage-renal-disease (ESRD) patients on dialysis treatment. It has been reported that more than 80% of young adults on dialysis already had severe and progressive coronary artery calcifications, as detected by electronic beam-computed tomography (EBCT). Hypercalcaemia, hyperphosphataemia as well as an increased calcium×phosphate (Ca×P) product level in serum are thought to be of pathophysiological relevance and associated with raised cardiovascular calcification in this patient population. However, severe ectopic or soft tissue calcifications including cardiovascular calcification have also been described independent of pronounced hypercalcaemia and/or hyperphosphataemia in patients on dialysis and in parathyroidectomised patients with uremia. In other words, determining calcium level, phosphate level and calcium×phosphate product level alone does not provide a sufficient method to identify or monitor a patient who has a risk of developing cardiovascular calcification.

Extracellular calcification inhibitors, also named as calcium regulatory proteins, including alpha-2 Heremans Schmid glycoprotein (alpha-2 HS glycoprotein, AHSG, Fetuin or Fetuin-A), Fetuin-B, matrix Gla protein (MGP), secreted phosphoprotein 24 (spp24), osteopontin, osteonectin, and bone morphogenetic protein-7 (BMP-7), etc. inhibit Ca×P precipitation. MGP may act locally as a potent inhibitor of aortic calcification. Correspondingly, MGP knockout mice develop severe arterial-media calcifications and die of aortic rupture at the age of 8 weeks.

AHSG has the highest known capacity in inhibiting soft tissue calcification among all other molecules in the circulation. It may be the most important and is a major calcification regulating protein in the circulation accounting for more than 50% of the total circulating calcification inhibitory capacity. AHSG knockout mice develop severe soft tissue and intravascular calcifications.

AHSG, a glycoprotein present in the circulation, is synthesized by hepatocytes. The AHSG molecule consists of two polypeptide chains, which are both cleaved from a proprotein encoded from a single mRNA. The protein is commonly present in the cortical plate of the immature cerebral cortex and bone marrow hemopoietic matrix. Under usual conditions, the circulating half-life of AHSG is estimated to be several days. However, its function of inhibiting soft tissue calcification is achieved by forming a soluble colloidal microsphere of fetuin-calcium-phosphate complex in the blood stream.

Recently, researchers have reported that AHSG deficiency is associated with inflammation and links vascular calcification to mortality in patients on dialysis. Activated acute-phase response and AHSG deficiency might account for accelerated atherosclerosis in uremia. It is also suggested that if uremic patients in the states of low AHSG level, therapeutic efforts would need to be intensified to prevent soft tissue calcification.

Ratios calculated from two measured values of different analytes has been used as a disease marker. It includes ratio of free prostate specific antigen (PSA) to total PSA, free PSA to alpha-1 antichymotrypsin (ACT) complex PSA, ACT complex PSA to total PSA, whole parathyroid hormone (PTH) to total intact PTH, N-truncated PTH to Total intact PTH, etc. U.S. Pat. Nos. 5,501,983 and 9,344,693 disclose examples of ratios as disease markers.

### SUMMARY OF THE INVENTION

The present invention includes methods of identifying or monitoring a patient with increased risk of cardiovascular calcification including measuring a human alpha-2 HS glycoprotein (AHSG, also named as Fetuin or Fetuin-A) level in a biological sample and comparing the AHSG level to at least one parameter selected from the group consisting of a calcium level, a phosphate level and a calcium×phosphate product level. The patient is determined to have or be at increased risk of developing cardiovascular calcification if the comparison value is different from that of normal population, a healthy control group or a control sample. The comparison may include calculating a ratio or proportion of ahpha-2 HS glycoprotein level to one or more calcification accelerating legends including but not limited to calcium, phosphate or calcium×phosphate product levels.

The present invention also includes methods of identifying or monitoring a patient with increased risk of cardiovascular calcification including determining a circulating calcification inhibitory capacity in a biological sample and comparing the capacity to at least one parameter selected from the group consisting of a calcium level, a phosphate level and a calcium×phosphate product level. The patient may be determined to have or be at increased risk of developing cardiovascular calcification if the comparison value is different from that of a normal population, a healthy control group or a control value.

The present invention also includes methods of monitoring the effectiveness of a patient's therapy or therapeutic treatment for a disease or condition suspected of effecting cardiovascular calcification. These methods include the measurement of AHSG or determination of the circulating calcification inhibitory capacity in a biological sample, comparing the measurement or capacity to at least one parameter selected from the group consisting of a calcium level, a phosphate level and a calcium×phosphate product level. One or more comparisons may be provided over time and may further be compared to one another, a normal population or a control value to determine whether treatment has a beneficial or detrimental effect on calcification.

### BRIEF DESCRIPTION OF THE DRAWINGS

FIG. 1 shows that the ratio of Ca×P to AHSG levels in normal population and patients with chronic kidney failure on dialysis treatment. A portion of dialysis patients had an elevated ratio of Ca×P to AHSG indicating that these group of patients may have an increased risk of ectopic calcification.

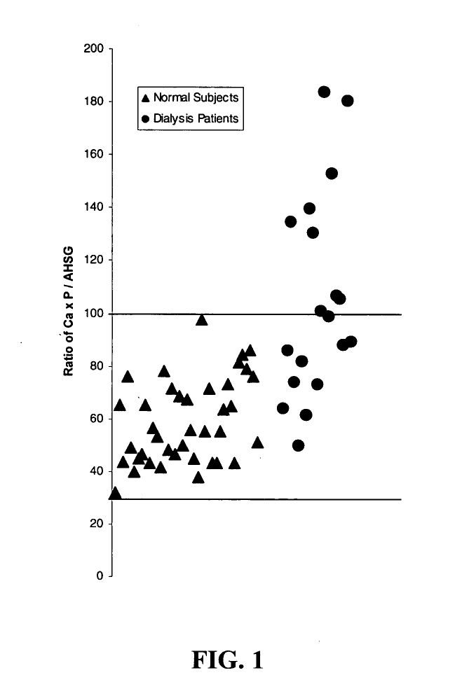

### DETAILED DESCRIPTION

The present invention may include one or more methods of identifying or monitoring a patient with increased risk of cardiovascular calcification including measuring an alpha-2 HS glycoprotein (AHSG) level or determining a calcification inhibitory capacity (CIC) in a biological sample and comparing the measurement or capacity to levels of factors or legends such as but not limited to calcium, phosphate or calcium×phosphate product (Ca×P). The AHSG and the CIC may represent the body's capability of preventing, inhibiting or reducing calcification (calcification inhibitory capacity) and it is preferable that the AHSG or CIC level is within the normal or desired range.

The normal range of AHSG or CIC may differ or vary depending on a particular population and may depend at least in part by the population's age, race, geographic region or genetic makeup. A control or normal population may be identified by choosing a population that has one or more characteristics similar to that of the patient such as the patient's age, race, geographical region, genetic makeup and the like. The normal range may then be calculated by pooling data obtained from two or more samples or individuals from the control, healthy or normal population and determining a mean or median value. A standard deviation may also be calculated. AHSG or CIC levels that are different such as substantially different or statistically different from the median or mean value or beyond one, two, three or more standard deviations may be deemed having or having a higher risk of developing cardiovascular calcification. As a non-limiting example, when measuring AHSG in a whole blood sample or a serum sample, a normal range of AHSG may be about 0.25 g/L, from about 0.25 g/L to about 0.50 g/L, from about 0.50 g/L to about 1 g/L or from about 1 g/L to about 2 g/L.

The factors or legends including but not limited to calcium, phosphate and a calcium×phosphate (Ca×P) product may contribute directly to the acceleration of cardiovascular calcification (also referred to as calcification accelerating legends (CAL)) when their levels or concentrations are abnormally high or in the range of an undesired level. A normal or desired range for each factor, legend or combination thereof may be obtained or identified by measuring the corresponding factors or legends in an appropriate normal, healthy or control population. An average value such as a mean or a median value may be obtained when two, three or more measurements are collected. In addition, a standard deviation may also be calculated. An undesired value or undesired level may be one that falls outside of a mean or median value or may be one that falls outside of one, two, three or more standard deviations from the mean or median value of the normal or control samples or population.

The undesired level of CAL may be different in various patient populations with various disease conditions. Therefore it may be desirable to obtain a normal level, range or control value from the appropriate patient population, which may or may not have a disease condition. An appropriate patient population may include individuals of the same sex, race or from the same geographical region and like when compared to the patient.

A ratio or proportion of calcification inhibitory capacity to calcification accelerating legends may be calculated to be a calcification risk index (CRI) that serves as a marker of the relative strength as well as the balance of preventing calcification vs. accelerating calcification. Non-limiting examples of mathematical formula of the ratio of CIC and CAL are listed as follow:

$$\begin{matrix}( 1 ) & Ratio of AHSG to Ca \times P Product \\ & R = \frac{Ca \times P \times a1}{AHSG \times b1}\end{matrix}$$

wherein a1 and b1 are given factors

$$\begin{matrix}( 2 ) & Ratio of AHSG to calcium \\ & R = \frac{Ca \times a1}{AHSG \times b1}\end{matrix}$$
wherein a1 and b1 are given factors

$$\begin{matrix}( 3 ) & Ratio of AHSG to phosphate \\ & R = \frac{P \times a1}{AHSG \times b1}\end{matrix}$$
wherein a1 and b1 are given factors

(4) Calculation of calcification inhibitory capacity (CIC)
CIC=k1×AHSG+k2×MGP+ . . . kn×Y
or
CIC=(k1×AHSG)×(k2×MGP)× . . . (kn×Y)
wherein k1, k2, . . . kn are given factors and Y is an analyte.

$$\begin{matrix}( 5 ) & Ratio of CIC to Ca \times P Product \\ & R = \frac{Ca \times P \times a1}{CIC \times b1}\end{matrix}$$
wherein a1 and b1 are given factors

$$\begin{matrix}( 6 ) & Ratio of CIC to calcium \\ & R = \frac{Ca \times a1}{CIC \times b1}\end{matrix}$$
wherein a1 and b1 are given factors

$$\begin{matrix}( 7 ) & Ratio of CIC to phosphate \\ & R = \frac{P \times a1}{CIC \times b1}\end{matrix}$$
wherein a1 and b1 are given factors

Alternatively, the above provided numerators and denominators may be reversed such that CIC or AHSG are the numerator. The equations may or may not include given factors. In equations 1-7 or equations derived from equations 1-7, one or more of the given factors are preferably one however this need not be the case. For example, the given value may take into account a variety of factors such as but not limited to the population size, age, race, geographic region or genetic makeup. The given value may also include a conversion factor.

The AHSG level or concentration in a biological sample may be determined by any appropriate quantitative measurement method such as but not limited to immunoassays including non-competitive “sandwich” immunoassay, competitive immunoassay, nephelometry or turbidimetry, etc. These immunoassay methods are documented and well know by those of ordinary skill in the art and may include but is not limited to the use of a monoclonal antibody against AHSG, a polyclonal antibody against AHSG, a molecule capable of binding AHSG and the like. Since the circulating AHSG may be in different forms such as free AHSG, AHSG mineral complex, AHSG MGP complex, AHSG spp24 complex, etc. different immunoassays for AHSG may measure the total AHSG including both free and complex forms or specifically measure one or several forms of AHSG such as AHSG mineral complex and/or AHSG MGP complex. Procedures for creating antibodies such as to AHSG or AHSG complexes may be found in a variety of laboratory manuals such as Antibodies, Cold Spring Harbor Laboratories (1988). In a preferred embodiment the total AHSG is measured. As non-limiting examples, a total AHSG concentration or level may include a free AHSG and a calcium, phosphate bounded AHSG, or an AHSG complex and a calcium, phosphate bounded AHSG complex, or a free AHSG and an AHSG complex, and a calcium, phosphate bounded free AHSG and AHSG complex.

The circulating calcification inhibitory capacity may be determined by calculating the sum value or the product value of at least two values, levels or concentrations of the calcification inhibitor levels selected from a group of AHSG, MGP, BMP-7, osteopontin, osteonectin, albumin and the like. The calcium and phosphate levels may be determined by a variety of routine clinical chemistry methods used in clinical laboratory practice such as but not limited to immunoassays including non-competitive “sandwich” immunoassay, competitive immunoassay, nephelometry, turbidimetry and the like. A calcium level or concentration may include a total calcium level, a corrected calcium level or an ionized calcium level.

Each of the disclosed analytes or compounds may be measured or detected from a variety of biological fluids or biological samples. For example, patient samples may include blood, whole blood, serum, sera and the like. The sample may be treated with one or more agents such as clotting agents or agents that prevent clotting.

One can compare the AHSG level or the calcification inhibitory capacity in a biological sample to calcium, phosphate or calcium×phosphate product level by using a ratio or proportion. The AHSG or CIC may be a denominator or a numerator. By doing so, one may identify or monitor a patient having increased risk of cardiovascular calcification, as well as to monitor an effect of treatment for a patient with increased risk of cardiovascular calcification or patient who already has cardiovascular calcification. Thus, one may be able to identify and monitor patient with increased risk of cardiovascular calcification, as well as to monitor an effect of treatment for patient with increased risk of cardiovascular calcification or patient who already has cardiovascular calcification.

The methods of the present invention also include monitoring the effectiveness of a therapeutic treatment in a patient for a disease or condition effecting cardiovascular calcification, which may include measuring a human alpha-2 HS glycoprotein (AHSG) level or concentration in a biological sample, comparing the AHSG level to at least one parameter selected from the group consisting of a calcium level, a phosphate level and a calcium×phosphate product level, repeating the measuring and comparing steps one or more times, and determining the therapeutic treatment is favorable if the comparison approaches or generally corresponds to a control value or a normal or healthy population value over a period of time. The present invention may be used to monitor the treatment of a variety of diseases such as but not limited to a chronic kidney disease, renal failure, uremia, diabetes, rheumatoid arthritis, a chronic liver disease, hepatitis and liver cirrhosis. Therapeutic treatment may then be adjusted in response to the patient's favorable or unfavorable progress.

The methods of the present invention also include monitoring the effectiveness of a therapeutic treatment in a patient for a disease or condition effecting cardiovascular calcification, which may include determining a circulating calcification inhibitory capacity in a biological sample, comparing the circulating calcification inhibitory capacity to at least one parameter selected from the group consisting of a calcium level, a phosphate level and a calcium×phosphate product level repeating the measuring and comparing steps one or more times over a period of time, and determining the therapeutic treatment is favorable if the comparison approaches a control value or a normal or healthy population value over the period of time. The present invention may be used to monitor the treatment of a variety of diseases such as but not limited to a chronic kidney disease, renal failure, uremia, diabetes, rheumatoid arthritis, a chronic liver disease, hepatitis and liver cirrhosis.

The present methods have been used in a clinical setting involving 79 persons. The group included 39 normal persons without any cardiovascular diseases, liver diseases, kidney diseases, mineral metabolism disorders, etc.; and 40 patients including 20 patients with end-stage renal disease on dialysis treatment and 20 patients with surgical proven primary hyperparathyroidism, a disease characterized with abnormal high levels of parathyroid hormone and calcium. Table 1 shows the results individually and comparatively, of the AHSG, calcium, phosphate, Ca×P product, as well as ratios from the normal populations.

TABLE 1
Control Population (Normal)

| Patient | | AHSG | Ca | P | | Ratio | Ratio | Ratio |
|---|---|---|---|---|---|---|---|---|
| No. | Sex | (g/l) | (mg/dl) | (mg/dl) | Ca × P | Ca/AHSG | P/AHSG | Ca × P/AHSG |
| 1 | M | 0.642 | 9.4 | 2.2 | 20.68 | 14.6 | 3.43 | 32.21 |
| 2 | F | 0.463 | 8.4 | 3.6 | 30.24 | 18.1 | 7.78 | 65.31 |
| 3 | F | 0.785 | 7.3 | 4.7 | 34.31 | 9.3 | 5.99 | 43.71 |
| 4 | F | 0.562 | 9.3 | 4.6 | 42.78 | 16.5 | 8.19 | 76.12 |
| 5 | F | 0.605 | 8.5 | 3.5 | 29.75 | 14.0 | 5.79 | 49.17 |
| 6 | F | 0.691 | 7.7 | 3.6 | 27.72 | 11.1 | 5.21 | 40.12 |
| 7 | F | 0.568 | 7.5 | 3.4 | 25.5 | 13.2 | 5.99 | 44.89 |
| 8 | F | 0.796 | 9.3 | 4.0 | 37.2 | 11.7 | 5.03 | 46.73 |
| 9 | F | 0.585 | 7.5 | 5.1 | 38.25 | 12.8 | 8.72 | 65.38 |
| 10 | F | 0.806 | 8.7 | 4.0 | 34.8 | 10.8 | 4.96 | 43.18 |
| 11 | F | 0.550 | 8.0 | 3.9 | 31.2 | 14.5 | 7.09 | 56.73 |
| 12 | F | 0.692 | 9.0 | 4.1 | 36.9 | 13.0 | 5.92 | 53.32 |
| 13 | F | 0.683 | 7.7 | 3.7 | 28.49 | 11.3 | 5.42 | 41.71 |
| 14 | F | 0.470 | 9.4 | 3.9 | 36.66 | 20.0 | 8.30 | 78.00 |
| 15 | F | 0.585 | 9.4 | 3.0 | 28.2 | 16.1 | 5.13 | 48.21 |
| 16 | M | 0.463 | 9.2 | 3.6 | 33.12 | 19.9 | 7.78 | 71.53 |
| 17 | F | 0.737 | 8.8 | 3.9 | 34.32 | 11.9 | 5.29 | 46.57 |
| 18 | F | 0.526 | 9.5 | 3.8 | 36.1 | 18.1 | 7.22 | 68.63 |
| 19 | F | 0.632 | 7.5 | 4.2 | 31.5 | 11.9 | 6.65 | 49.84 |
| 20 | M | 0.436 | 8.9 | 3.3 | 29.37 | 20.4 | 7.57 | 67.36 |
| 21 | F | 0.552 | 8.8 | 3.5 | 30.8 | 15.9 | 6.34 | 55.80 |
| 22 | F | 0.506 | 7.8 | 2.9 | 22.62 | 15.4 | 5.73 | 44.70 |
| 23 | F | 0.927 | 9.0 | 3.9 | 35.1 | 9.7 | 4.21 | 37.86 |
| 24 | F | 0.451 | 9.0 | 4.9 | 44.1 | 20.0 | 10.86 | 97.78 |
| 25 | F | 0.527 | 9.4 | 3.1 | 29.14 | 17.8 | 5.88 | 55.29 |
| 26 | F | 0.496 | 7.7 | 4.6 | 35.42 | 15.5 | 9.27 | 71.41 |
| 27 | F | 0.680 | 8.9 | 3.3 | 29.37 | 13.1 | 4.85 | 43.19 |
| 28 | F | 0.688 | 8.5 | 3.5 | 29.75 | 12.4 | 5.09 | 43.24 |
| 29 | F | 0.520 | 9.3 | 3.1 | 28.83 | 17.9 | 5.96 | 55.44 |
| 30 | F | 0.548 | 8.7 | 4.0 | 34.8 | 15.9 | 7.30 | 63.50 |
| 31 | F | 0.412 | 9.4 | 3.2 | 30.08 | 22.8 | 7.77 | 73.01 |
| 32 | F | 0.492 | 7.1 | 4.5 | 31.95 | 14.4 | 9.15 | 64.94 |
| 33 | M | 0.730 | 9.6 | 3.3 | 31.68 | 13.2 | 4.52 | 43.40 |
| 34 | F | 0.480 | 9.3 | 4.2 | 39.06 | 19.4 | 8.75 | 81.38 |
| 35 | F | 0.424 | 9.4 | 3.8 | 35.72 | 22.2 | 8.96 | 84.25 |
| 36 | F | 0.394 | 8.2 | 3.8 | 31.16 | 20.8 | 9.64 | 79.09 |
| 37 | F | 0.406 | 9.2 | 3.8 | 34.96 | 22.7 | 9.36 | 86.11 |
| 38 | F | 0.429 | 8.6 | 3.8 | 32.68 | 20.0 | 8.86 | 76.18 |
| 39 | M | 0.718 | 9.2 | 4.0 | 36.8 | 12.8 | 5.57 | 51.25 |

| | | | | | | | |
|---|---|---|---|---|---|---|---|
| Mean | 0.6 | 8.7 | 3.8 | 32.6 | 15.7 | 6.8 | 58.9 |
| Median | 0.6 | 8.9 | 3.8 | 32.0 | 15.4 | 6.3 | 55.4 |
| SD | 0.1 | 0.7 | 0.6 | 4.8 | 3.8 | 1.8 | 16.0 |

Table 2 shows the results individually and comparatively, of the AHSG, calcium, phosphate, Ca×P product, as well as ratios from dialysis patients.

TABLE 2
Patient's With End Stage Renal Disease

| Patient | | AHSG | Ca | P | | Ratio | Ratio | Ratio |
|---|---|---|---|---|---|---|---|---|
| No. | Sex | (g/l) | (mg/dl) | (mg/dl) | Ca × P | Ca/AHSG | P/AHSG | Ca × P/AHSG |
| 41 | F | 0.562 | 7.2 | 5.0 | 36 | 12.8 | 8.90 | 64.06 |
| 42 | M | 0.526 | 7.8 | 5.8 | 45.24 | 14.8 | 11.03 | 86.01 |
| 43 | F | 0.290 | 6.2 | 6.3 | 39.06 | 21.4 | 21.72 | 134.69 |
| 44 | F | 0.532 | 6.9 | 5.7 | 39.33 | 13.0 | 10.71 | 73.93 |
| 45 | M | 0.573 | 9.2 | 3.1 | 28.52 | 16.1 | 5.41 | 49.77 |
| 46 | F | 0.418 | 7.3 | 4.7 | 34.31 | 17.5 | 11.24 | 82.08 |
| 47 | F | 0.470 | 6.9 | 4.2 | 28.98 | 14.7 | 8.94 | 61.66 |
| 48 | F | 0.321 | 8.0 | 5.6 | 44.8 | 24.9 | 17.45 | 139.56 |
| 49 | M | 0.313 | 8.7 | 4.7 | 40.89 | 27.8 | 15.02 | 130.64 |
| 50 | F | 0.452 | 7.5 | 4.4 | 33 | 16.6 | 9.73 | 73.01 |
| 51 | F | 0.275 | 8.7 | 3.2 | 27.84 | 31.6 | 11.64 | 101.24 |
| 52 | F | 0.470 | 8.3 | 10.4 | 86.32 | 17.7 | 22.13 | 183.66 |
| 54 | M | 0.344 | 8.3 | 4.1 | 34.03 | 24.1 | 11.92 | 98.92 |
| 55 | M | 0.500 | 8.6 | 8.9 | 76.54 | 17.2 | 17.80 | 153.08 |
| 56 | M | 0.473 | 9.9 | 5.1 | 50.49 | 20.9 | 10.78 | 106.74 |
| 57 | M | 0.506 | 6.6 | 8.1 | 53.46 | 13.0 | 16.01 | 105.65 |
| 58 | F | 0.464 | 7.3 | 5.6 | 40.88 | 15.7 | 12.07 | 88.10 |
| 59 | M | 0.320 | 8.5 | 6.8 | 57.8 | 26.6 | 21.25 | 180.63 |
| 60 | M | 0.605 | 9.5 | 5.7 | 54.15 | 15.7 | 9.42 | 89.50 |

| | | | | | | | |
|---|---|---|---|---|---|---|---|
| Mean | 0.443 | 7.968 | 5.653 | 44.823 | 19.058 | 13.324 | 105.42 |
| Median | 0.470 | 8.000 | 5.600 | 40.880 | 17.200 | 11.636 | 98.92 |
| SD | 0.103 | 1.016 | 1.856 | 15.696 | 5.550 | 4.812 | 38.74 |

Using AHSG alone and assuming a cutoff or a normal level or concentration of about 0.4 g/L, about 30% of the patients depicted in Table 2 would have been identified as having high risk of cardiovascular calcification since their AHSG level or concentration is below 0.4 g/L, or two standard deviations away from the mean value of 0.6 g/L.

However, if the comparisons or ratios disclosed in the present invention are used, more patients, potentially about 50%, would have high risk of soft tissue calcification. For example if a normal range of the Ca×P/AHSG ratio has an upper limit of about 90 then a patient having a ratio over 90 may be considered at higher risk of having or developing soft-tissue calcification than a patient with a ratio below 90. Similarly if a normal range upper limit or cut-off of the P/AHSG ratio is 10 then a patient with a P/AHSG ratio above 10 may be considered at higher risk of having or developing soft-tissue calcification than a patient having a P/AHSG ration below 10. An imbalanced Ca×P/AHSG ratio or the relative strength of CIC to CAL may lead to increased risk of soft tissue calcification and the increased Ca×P/AHSG ratio may caused by (1) an abnormally higher Ca×P product level with relatively normal AHSG level, such as patient #52 and #55 in the table 2; (2) an abnormally deficiency of serum AHSG level, although these patients had a relatively normal Ca×P product level (patients # 43, 48, 49, 51 in the table 2) and (3) patients with both abnormally decreased AHSG level and abnormally increased Ca×P product level (patient # 59 in the table 2).

Table 3 shows the results individually and comparatively, of the AHSG, calcium, phosphate, Ca×P product, as well as ratios from patients with primary hyperparathyroidism.

TABLE 3
Patients with Surgical Proven Primary Hyperparathyroidism

| Patient | | AHSG | Ca | P | | Ratio | Ratio | Ratio |
|---|---|---|---|---|---|---|---|---|
| No. | Sex | (g/l) | (mg/dl) | (mg/dl) | Ca × P | Ca/AHSG | P/AHSG | Ca × P/AHSG |
| 61 | F | 0.35 | 10.7 | 3 | 32.1 | 30.57 | 8.57 | 91.71 |
| 62 | F | 0.52 | 12.1 | 3.1 | 37.51 | 23.27 | 5.96 | 72.13 |
| 63 | F | 0.44 | 10.5 | 3.1 | 32.55 | 23.86 | 7.05 | 73.98 |
| 64 | F | 0.34 | 11 | 2.9 | 31.9 | 32.35 | 8.53 | 93.82 |
| 65 | F | 0.46 | 11 | 2.3 | 25.3 | 23.91 | 5.00 | 55.00 |
| 66 | F | 0.41 | 11.2 | 4.4 | 49.28 | 27.32 | 10.73 | 120.20 |
| 67 | F | 0.32 | 11 | 2.9 | 31.9 | 34.38 | 9.06 | 99.69 |
| 68 | F | 0.47 | 10.8 | 2.6 | 28.08 | 22.98 | 5.53 | 59.74 |
| 69 | F | 0.33 | 11 | 2.1 | 23.1 | 33.33 | 6.36 | 70.00 |
| 70 | M | 0.57 | 10.5 | 2.4 | 25.2 | 18.42 | 4.21 | 44.21 |
| 71 | F | 0.54 | 10.5 | 3.4 | 35.7 | 19.44 | 6.30 | 66.11 |
| 72 | F | 0.55 | 10.7 | 3.1 | 33.17 | 19.45 | 5.64 | 60.31 |
| 73 | M | 0.44 | 10.1 | — | 0 | 22.95 | 0.00 | 0.00 |
| 74 | F | 0.63 | 10.8 | 3.4 | 36.72 | 17.14 | 5.40 | 58.29 |
| 75 | F | 0.47 | 10.9 | 2.8 | 30.52 | 23.19 | 5.96 | 64.94 |
| 76 | F | 0.43 | 10.2 | 3.2 | 32.64 | 23.72 | 7.44 | 75.91 |
| 77 | M | 0.65 | 10.6 | 3.5 | 37.1 | 16.31 | 5.38 | 57.08 |
| 78 | F | 0.43 | 10.7 | 3.2 | 34.24 | 24.88 | 7.44 | 79.63 |
| 79 | F | 0.46 | 11.4 | 2.4 | 27.36 | 24.78 | 5.22 | 59.48 |
| 80 | F | 0.68 | 10.8 | 3.4 | 36.72 | 15.88 | 5.00 | 54.00 |

| | | | | | | | |
|---|---|---|---|---|---|---|---|
| Mean | 0.47 | 10.83 | 3.01 | 31.05 | 23.91 | 6.24 | 67.81 |
| Median | 0.46 | 10.80 | 3.10 | 32.33 | 23.50 | 5.96 | 65.52 |
| SD | 0.10 | 0.43 | 0.53 | 9.28 | 5.47 | 2.20 | 24.27 |

The statistically significant differences in the averages between the dialysis patient group and the normal group, as well as the 30% of individuals having an abnormally low level of AHSG within a dialysis patient group and there are only 4 individuals showing a lower than normal low cut off level such as 0.4 g/l in patients with primary hyperparathyroidism (Table 4) demonstrates that one can identify and monitor patient with increased risk of cardiovascular calcification with AHSG level alone. However, a ratio of Ca×P/AHSG or P/AHSG is a much better index to identify more renal failure patients with increased risk of calcification (50% using Ca×P/AHSG and 70% using P/AHSG) and to differentiate these patients better from normal population as well as patients with metabolic disease such as primary hyperparathyroidism. Moreover, these ratios may be also better indices to monitor the effects of treatment for a patient with increased risk of cardiovascular calcification or patient who already has cardiovascular calcification.

TABLE 4

| | AHSG | Ca | P | | Ratio | Ratio | Ratio |
|---|---|---|---|---|---|---|---|
| | (g/l) | (mg/dl) | (mg/dl) | Ca × P | Ca/AHSG | P/AHSG | Ca × P/AHSG |
| Normal Subjects, n = 39 | 0.6 ± 0.1 | 8.7 ± 0.7 | 3.8 ± 0.6 | 32.6 ± 4.8 | 15.7 ± 3.8 | 6.8 ± 1.8 | 58.9 ± 16.0 |
| Dialysis Patients, n = 20 | 0.4 ± 0.1 | 8.0 ± 1.0 | 5.7 ± 1.9 | 44.8 ± 15.7 | 19.1 ± 5.6 | 13.3 ± 4.8 | 105.4 ± 38.7 |
| Primary | 0.5 ± 0.1 | 10.8 ± 0.4 | 3.0 ± 0.5 | 31.1 ± 9.3 | 23.9 ± 5.5 | 6.2 ± 2.2 | 67.8 ± 24.3 |
| Hyperparathyroidism | | | | | | | |
| Patients, n = 20 | | | | | | | |

The present method has also been used in another clinical setting involving 214 patients with end stage renal disease. This group of patients was followed up for six years with 53 patients dying because of cardiovascular event, which related to some degree of cardiovascular calcification. The total mortality was about 24.8 percent. Table 5 shows that the CRI, which is calculated as a ratio of AHSG and Ca×P product, is positively correlated to patient mortality. However, a negative correlation was observed with serum AHSG level, but not the Ca×P product level.

TABLE 5

| | Number of Patients | | Survived | Dead | Mortality |
|---|---|---|---|---|---|
| CRI (Ca × | | | | | |
| P/AHSG) | | | | | |
| <100 | 20/214 | (9.3%) | 17 | 3 | 15% |
| 100-200 | 66/214 | (30.8%) | 54 | 12 | 18.2% |
| 200-300 | 47/214 | (22.0%) | 40 | 7 | 15% |
| 300-400 | 32/214 | (15.0% | 22 | 10 | 31.3% |
| 400-500 | 25/214 | (11.7%) | 17 | 8 | 32% |
| >500 | 24/214 | (11.2%) | 11 | 13 | 54.2% |
| | | | 161 | 53 | 24.8% |
| AHSG(g/L) | | | | | |
| >0.4 | 48/214 | (22.4%) | 41 | 7 | 14.6% |
| 0.3-0.4 | 24/214 | (11.2%) | 19 | 5 | 20.8% |
| 0.2-0.3 | 63/214 | (29.4%) | 53 | 10 | 15.9% |
| 0.1-0.2 | 62/214 | (29.0%) | 42 | 21 | 33.9% |
| <0.1 | 16/214 | (7.5%) | 6 | 10 | 62.5% |
| | | | 161 | 53 | 24.8% |
| Ca × P | | | | | |
| <30 | 7/214 | (3.3%) | 5 | 2 | 28.6% |
| 30-45 | 29/214 | (13.6%) | 22 | 7 | 24.1% |
| 45-55 | 58/214 | (27.1%) | 42 | 16 | 27.6% |
| 55-65 | 47/214 | (22.0%) | 37 | 10 | 21.3% |
| 65-75 | 32/214 | (15.0%) | 25 | 7 | 21.9% |
| >75 | 41/214 | (19.2%) | 30 | 11 | 26.8% |
| | | | 161 | 53 | 24.8% |

The ordinarily skilled artisan can appreciate that the present invention includes any number of the preferred aspects and embodiments described above. Other embodiments of the present invention that are not presented here, which are or would be obvious to those of ordinary skill in the art, now or during the term of any patent issuing from this specification that are within the spirit and scope of the present invention are encompassed by the present invention.

All publications or unpublished patent applications mentioned or cited in the present invention and application are incorporated by reference in their entirety.

### REFERENCES

1. Schafer C, Heiss A, Schwarz A, Westenfeld R, Ketteler M, Floege J, Muller-Esterl W, Schinke T, Jahnen-Dechent W. The serum protein alpha 2-Heremans-Schmid glycoprotein/fetuin-A is a systemically acting inhibitor of ectopic calcification. J Clin Invest. 2003 August; 112(3):357-66.
2. Ketteler M, Vermeer C, Wanner C, Westenfeld R, Jahnen-Dechent W, Floege J. Novel insights into uremic vascular calcification: role of matrix Gla protein and alpha-2-Heremans Schmid glycoprotein/fetuin. Blood Purif. 2002; 20(5):473-6. Review.
3. Ketteler M, Bongartz P, Westenfeld R, Wildberger J E, Mahnken A H, Bohm R, Metzger T, Wanner C, Jahnen-Dechent W, Floege J. Association of low fetuin-A (AHSG) concentrations in serum with cardiovascular mortality in patients on dialysis: a cross-sectional study. Lancet. 2003 Mar. 8; 361(9360):827-33.
4. Ketteler M, Wanner C, Metzger T, Bongartz P, Westenfeld R, Gladziwa U, Schurgers L J, Vermeer C, Jahnen-Dechent W, Floege J. Deficiencies of calcium-regulatory proteins in dialysis patients: a novel concept of cardiovascular calcification in uremia. Kidney Int Suppl. 2003 May; (84):S84-7. Review.
5. Jahnen-Dechent W, Schafer C, Heiss A, Grotzinger J. Systemic inhibition of spontaneous calcification by the serum protein alpha 2-HS glycoprotein/fetuin. Z. Kardiol. 2001; 90 Suppl 3:47-56.
6. Jahnen-Dechent W, Schinke T, Trindl A, Muller-Esterl W, Sablitzky F, Kaiser S, Blessing M. Cloning and targeted deletion of the mouse fetuin gene. J Biol Chem. 1997 Dec. 12; 272(50):31496-503.
7. Heiss A, DuChesne A, Denecke B, Grotzinger J, Yamamoto K, Renne T, Jahnen-Dechent W. Structural basis of calcification inhibition by alpha 2-HS glycoprotein/fetuin-A. Formation of colloidal calciprotein particles. J Biol Chem. 2003 Apr 11; 278(15):13333-41. Epub 2003 Jan 29.
8. Strumia R, Lombardi A R, Bedani P L, Perini L. Benign nodular calcification and calciphylaxis in a haemodialysed patient. J Eur Acad Dermatol Venereol. 1998 July; 11(1):69-71.
9. Steitz S A, Speer M Y, McKee M D, Liaw L, Almeida M, Yang H, Giachelli C M. Osteopontin inhibits mineral deposition and promotes regression of ectopic calcification. Am J Pathol. 2002 December; 161(6):2035-46.
10. Milliner D S, Zinsmeister A R, Lieberman E, Landing B. Soft tissue calcification in pediatric patients with end-stage renal disease. Kidney Int. 1990 November; 38(5):931-6.
11. Davies M R, Lund R J, Hruska K A. BMP-7 is an efficacious treatment of vascular calcification in a murine model of atherosclerosis and chronic renal failure. J Am Soc Nephrol. 2003 June; 14(6):1559-67.
12. Speer M Y, McKee M D, Guldberg R E, Liaw L, Yang H Y, Tung E, Karsenty G, Giachelli CM. Inactivation of the osteopontin gene enhances vascular calcification of matrix Gla protein-deficient mice: evidence for osteopontin as an inducible inhibitor of vascular calcification in vivo. J Exp Med. 2002 Oct. 21; 196(8):1047-55.
13. Munoz S J, Nagelberg S B, Green P J, Angstadt J D, Yang S L, Jarrell B E, Maddrey W C. Ectopic soft tissue calcium deposition following liver transplantation. Hepatology. 1988 May-June; 8(3):476-83.
14. Janigan D T, Hirsch D J, Klassen G A, MacDonald A S. Calcified subcutaneous arterioles with infarcts of the subcutis and skin (“calciphylaxis”) in chronic renal failure. Am J Kidney Dis. 2000 April; 35(4):588-97. Review.
15. Kerr D N. Hypercalcemia and metastatic calcification. Cardiovasc Res. 1997 December; 36(3):293-7. Review. No abstract available.
16. Klaassen-Broekema N, van Bijsterveld O P. A local challenger of ocular calciphylaxis in patients with chronic renal failure: a hypothesis. Graefes Arch Clin Exp Ophthalmol. 1995 November; 233(11):717-20.
17. Wilmer W A, Magro C M. Calciphylaxis: emerging concepts in prevention, diagnosis, and treatment. Semin Dial. 2002 May-June; 15(3):172-86. Review.
18. Block G A. Control of serum phosphorus: implications for coronary artery calcification and calcific uremic arteriolopathy (calciphylaxis). Curr Opin Nephrol Hypertens. 2001 November; 10(6):741-7. Review.
19. Bostrom K. Insights into the mechanism of vascular calcification. Am J Cardiol. 2001 Jul. 19; 88(2A):20E-22E.
20. Davies M R, Hruska K A. Pathophysiological mechanisms of vascular calcification in end-stage renal disease. Kidney Int. 2001 August; 60(2):472-9. Review.
21. Giachelli C M. Ectopic calcification: new concepts in cellular regulation. Z Kardiol. 2001; 90 Suppl 3:31-7.
22. Price P A, Williamson M K, Minh Thi Nguyen T, Than T N. Serum levels of the fetuin-mineral complex correlate with artery calcification in the rat. J Biol Chem. 2003 Oct 24 [Epub ahead of print]
23. Joshi R R, Underwood T, Frautschi J R, Phillips R E Jr, Schoen F J, Levy R J. Calcification of polyurethanes implanted subdermally in rats is enhanced: by calciphylaxis. J Biomed Mater Res. 1996 June; 31(2):201-7.
24. Luo G, Ducy P, McKee M D, Pinero G J, Loyer E, Behringer R R, Karsenty G. Spontaneous calcification of arteries and cartilage in mice lacking matrix GLA protein. Nature. 1997 Mar. 6; 386(6620):78-81.

1. A method for identifying or monitoring a patient with increased risk of cardiovascular calcification comprising:
a) measuring a human alpha-2 HS glycoprotein (AHSG) level in a biological sample;
b) comparing said AHSG level to at least one parameter selected from the group consisting of a calcium level, a phosphate level and a calcium×phosphate product level; and
c) determining said patient has or is at increased risk of developing cardiovascular calcification if said comparison is different from a normal range of a normal population or a healthy control group.

2. The method of claim 1, wherein said patient has at least one disease or condition selected from the group consisting of a chronic kidney disease, renal failure, uremia, diabetes, rheumatoid arthritis, a chronic liver disease, hepatitis and liver cirrhosis.

3. The method of claim 1, wherein said AHSG level is determined by a quantitative immunological method selected from the group consisting of a non-competitive “sandwich” immunoassay, a competitive immunoassay, a radial immunodiffusion method, a turbidimetry method and a nephelometry method.

4. The method of claim 1, wherein said AHSG level is a total AHSG level comprising:
a) a free AHSG and a calcium, phosphate bounded AHSG; or
b) an AHSG complex and a calcium, phosphate bounded AHSG complex; or
c) a free AHSG and an AHSG complex, and a calcium, phosphate bounded free AHSG and AHSG complex.

5. The method of claim 4, wherein said AHSG complex is an AHSG-MGP (Matrix Gla Protein) complex or an AHSG-spp24 (secreted phosphoprotein 24) complex.

6. The method of claim 1, wherein said comparison is in the form of a ratio or a proportion.

7. The method of claim 6, wherein said ratio or said proportion is selected from the group consisting of (Calcium level×Phosphate level)/AHSG, (Phosphate level)/AHSG and (Calcium Level)/AHSG; AHSG/(Calcium level×Phosphate level), AHSG/(Phosphate level) and AHSG/(Calcium Level).

8. The method of claim 1, wherein said calcium level is selected from the group consisting of a total calcium level, a corrected calcium level and an ionized calcium level.

9. A method for identifying or monitoring a patient with increased risk of cardiovascular calcification comprising:
a) determining a circulating calcification inhibitory capacity in a biological sample;
b) comparing said circulating calcification inhibitor capacity to at least one parameter selected from the group consisting of a calcium level, a phosphate level and a calcium×phosphate product level; and
c) determining said patient has or is at increased risk of developing cardiovascular calcification if said comparison is different from a normal population or a control value.

10. The method of claim 9, wherein said patient has at least one disease or condition selected from the group consisting of a chronic kidney disease, renal failure, uremia, diabetes, rheumatoid arthritis, a chronic liver disease, hepatitis and liver cirrhosis.

11. The method of claim 9, wherein said circulating calcification inhibitory capacity is the sum or product level of at least two values from at least two calcification inhibitors selected from the group consisting of AHSG, matrix Gla Protein (MGP), secreted phosphoprotein 24 (spp24), osteopontin, bone morphogenetic protein 7 (BMP-7), albumin and osteonectin.

12. The method of claim 9, wherein said calcium level is selected from the group consisting of a total calcium level, a corrected calcium level and an ionized calcium level.

13. A method for monitoring the effectiveness of a therapeutic treatment in a patient for a disease or condition effecting cardiovascular calcification comprising:
a) measuring a human alpha-2 HS glycoprotein (AHSG) level in a biological sample;
b) comparing said AHSG level to at least one parameter selected from the group consisting of a calcium level, a phosphate level and a calcium×phosphate product level;
c) repeating steps a) and b) one or more times over a period of time; and
d) determining said therapeutic treatment is favorable if said comparison approaches a control value or a normal population value over said period of time.

14. The method of claim 13, wherein said disease or condition is selected from the group consisting of a chronic kidney disease, renal failure, uremia, diabetes, rheumatoid arthritis, a chronic liver disease, hepatitis and liver cirrhosis.

15. The method of claim 13, wherein said human AHSG level is determined by a quantitative immunological method selected from the group consisting of non-competitive “sandwich” immunoassay, competitive immunoassay, radial immunodiffusion method, a turbidimetry method and nephelometry method.

16. The method of claim 15, wherein said AHSG level is a total AHSG level comprising:
a) a free AHSG and a calcium, phosphate bounded AHSG; or
b) an AHSG complex and a calcium, phosphate bounded AHSG complex; or
c) a free AHSG and an AHSG complex, and a calcium, phosphate bounded free AHSG and AHSG complex.

17. The method of claim 15, wherein said comparison is in the form of a ratio or a proportion.

18. The method of claim 17, wherein said ratio or said proportion is selected from the group consisting of (Calcium level×Phosphate level)/AHSG, (Phosphate level)/AHSG and (Calcium level)/AHSG; AHSG/(Calcium level×Phosphate level), AHSG/(Phosphate level) and AHSG/(Calcium Level).

19. The method of claim 13, wherein said calcium level is selected from the group consisting of a total calcium level, a corrected calcium level and an ionized calcium level.

20. A method for monitoring the effectiveness of a therapeutic treatment in a patient for a disease or condition effecting cardiovascular calcification comprising:
a) determining a circulating calcification inhibitory capacity in a biological sample;
b) comparing said circulating calcification inhibitory capacity to at least one parameter selected from the group consisting of a calcium level, a phosphate level and a calcium×phosphate product level; and
c) repeating steps a) and b) one or more times over a period of time; and
d) determining said therapeutic treatment is favorable if said comparison approaches a control value or a normal population value over said period of time.

21. The method of claim 20, wherein said circulating calcification inhibitory capacity is the sum or product level of at least two values from at least two calcification inhibitors selected from the group consisting of AHSG, MGP, spp24, osteopontin, BMP-7, albumin and osteonectin.

22. The method of claim 20, wherein the calcium level is selected from the group consisting of a total calcium level, a corrected calcium level and an ionized calcium level.
